(19) United States
(12) Patent Application Publication
(10) Pub. No.: US 2024/0177213 A1
(43) Pub. Date: May 30, 2024

(54) APPARATUS AND METHOD FOR RECOMMENDING CUSTOMIZED COSMETICS BASED ON ARTIFICIAL INTELLIGENCE

(71) Applicant: IBT Co.,Ltd, Cheongju-si (KR)

(72) Inventor: Jeonghun YOON, Sejong-si (KR)

(73) Assignee: IBT Co.,Ltd, Cheongju-si (KR)

(21) Appl. No.: 18/378,477

(22) Filed: Oct. 10, 2023

(30) Foreign Application Priority Data
Nov. 24, 2022 (KR) 10-2022-0159488

(51) Int. Cl. G06Q 30/0601

(52) U.S. Cl. CPC G06Q 30/0631; G06Q 30/0621

(57) ABSTRACT

An apparatus, for recommending customized cosmetics based on artificial intelligence, includes: a user skin information receiver for receiving information about a user's skin including a skin image and questionnaire data; a skin determination unit for determining skin type and skin condition by analyzing information about the user's skin; a skin-tailored formula generator for determining customized ingredients to be added and the additional ingredient content based on the skin type and the skin condition to generate information about a skin-tailored formula; and a customized cosmetics recommendation unit for providing information about customized cosmetics that recommends at least one of existing cosmetic products pre-registered for each skin type as a base cosmetic product based on the skin type and provides information on user-customized cosmetics by combining a skin-tailored formula with the recommended base cosmetic product.

### CROSS-REFERENCE TO PRIOR APPLICATION

This application claims priority to Korean Patent Application No. 10-2022-0159488 (filed on Nov. 24, 2022), which is hereby incorporated by reference in its entirety.

### BACKGROUND

The present disclosure relates to technology for recommending customized cosmetics based on artificial intelligence, and, more specifically, to an apparatus and a method for recommending customized cosmetics optimized for the skin condition of a user's scalp, face, body, etc. based on artificial intelligence analysis.

Recently, consumers prefer their own customized products rather than standardized and ready-made products targeting unspecified customers. Accordingly, the ministry of food and drug safety has reflected this trend in cosmetic products and begun implementing the “personalized cosmetics” system to provide cosmetics tailored to each individual customer's skin characteristics and tastes.

Customized cosmetics refers to a method of creating new cosmetics by adding/mixing the contents or raw materials of other cosmetics to cosmetics that have been already prepared or providing some of the contents of cosmetics that have been already prepared. The overall process for customized cosmetics consists of four steps: skin diagnosis and evaluation, research on materials and prescriptions, manufacturing method, and packaging and sales.

In general, to prepare and supply cosmetics, skin color, the amount of moisture in the skin, the amount of oil in the skin, elasticity, wrinkles, pigmentation, the number of pores, dead skin cells, skin texture, sensitivity, etc. are evaluated by surveys, instrumental skin diagnosis, and genetic analysis data, and a skin-tailored prescription is given. However, although self-diagnosis based on a survey is simple, it is difficult to accurately evaluate the skin. In addition, in the case of instrumental skin diagnosis and genetic diagnosis, it is possible to accurately evaluate the skin based on scientific evidence, but there is the inconvenience of having to have necessary equipment and collect skin cells, blood, saliva, etc. required for genetic analysis.

Therefore, there is a need for a system for allowing users to easily determine their skin condition and purchase customized cosmetics.

### Related Art Literature

(Patent Literature 1) Korean Patent Publication No. 10-0708319 (Apr. 10, 2007)
(Patent Literature 2) Korean Patent Application Publication No. 10-2022-0026468 (Mar. 4, 2022)

### SUMMARY

An embodiment of the present disclosure is aimed at providing an apparatus and a method for recommending customized cosmetics optimized for the skin condition of a user's scalp, face, body, etc. based on artificial intelligence analysis.

An embodiment of the present disclosure is aimed at providing an apparatus and a method for recommending customized cosmetics based on artificial intelligence by analyzing the skin based on a user's skin image and recommending ready-made cosmetic products based on the analysis results or customized cosmetics obtained by combining a formula for active ingredients with base cosmetics.

An embodiment of the present disclosure is aimed at providing an apparatus and a method for recommending customized cosmetics based on artificial intelligence by transmitting information on a formula for a base and additives of customized cosmetics recommended based on a user's order request to a cosmetics producer to provide a customized service for cosmetics to the user.

According to an embodiment of the present disclosure, an apparatus for recommending customized cosmetics based on artificial intelligence may include: a user skin information receiver for receiving information about a user's skin including a skin image and questionnaire data; a skin determination unit for determining skin type and skin condition by analyzing information about the user's skin; a skin-tailored formula generator for determining customized ingredients to be added and the additional ingredient content based on the skin type and the skin condition to generate information about a skin-tailored formula; and a customized cosmetics recommendation unit for providing information about customized cosmetics that recommends at least one of existing cosmetic products pre-registered for each skin type as a base cosmetic product based on the skin type and provides information on user-customized cosmetics by combining a skin-tailored formula with the recommended base cosmetic product.

The skin determination unit may determine at least one candidate group of skin types by analyzing a user's subjective skin condition based on the questionnaire data and finally determine the user's skin type and skin condition by analyzing the user's objective skin condition based on the skin image.

Based on an artificial intelligence analysis model for skin diagnosis that has been pre-built by artificial intelligence learning based on a skin image, the skin determination unit may analyze the received skin image for oiliness, dandruff, dead skin cells, erythema, and hair loss when a target skin is scalp, for oiliness, whitening, skin sensitivity, and wrinkles when a target skin is a face, and for oiliness, whitening, and skin sensitivity when a target skin is a body.

The customized cosmetics recommendation unit may set the range of skin types to which existing cosmetic products can be applied, recommend at least one existing cosmetic product set in a range suitable for a user's skin type as a base cosmetic product among pre-registered existing cosmetic products, and recommend an existing cosmetic product set in a close range as a base cosmetic product when there are no cosmetic products set in a range suitable for the user's skin type.

The customized cosmetics recommendation unit may provide a user with customized cosmetics prepared by mixing active ingredients with a recommended base cosmetic product based on the information on the skin-tailored formula or provide the base cosmetic product and ampoule containing the active ingredients for the user to mix them.

According to an embodiment of the present disclosure, a method of recommending customized cosmetics based on artificial intelligence may involve: receiving information about a user's skin including a skin image and questionnaire data for the user; determining a candidate group of skin types based on the questionnaire data and analyzing the received skin image based on a pre-constructed artificial intelligence analysis model for skin diagnosis to finally determine skin type and skin condition; generating information on a skin-tailored formula by determining additional ingredients and the additional ingredient content based on the finally determined skin type and skin condition; and recommending at least one of pre-registered existing cosmetic products for each skin type as a base cosmetic product based on the skin type and providing the information about the skin-tailored formula to be combined with the recommended base cosmetic product, allowing a cosmetics producer to provide a user with a customized service for cosmetics.

The present disclosure may have the following effects. However, it does not mean that a specific embodiment should include all of the following effects or only the following effects, so the scope of the present disclosure should not be deemed to be limited thereto.

According to an embodiment of the present disclosure, it may be possible for the apparatus and the method for recommending customized cosmetics based on artificial intelligence to recommend customized cosmetics optimized for the skin condition of a user's scalp, face, body, etc. based on artificial intelligence analysis.

According to an embodiment of the present disclosure, it may be possible for the apparatus and the method for recommending customized cosmetics based on artificial intelligence to analyze the skin based on a user's skin image and recommend ready-made cosmetic products based on the analysis results or customized cosmetics obtained by combining a formula for active ingredients with base cosmetics.

According to an embodiment of the present disclosure, it may be possible for the apparatus and the method for recommending customized cosmetics based on artificial intelligence to transmit information on a formula for a base and additives of customized cosmetics recommended based on a user's order request to a cosmetics producer to provide the user with a customized service for cosmetics.

### BRIEF DESCRIPTION OF THE DRAWINGS

FIG. 1 shows a system for recommending customized cosmetics based on artificial intelligence according to an embodiment of the present disclosure.

FIG. 2 is a block diagram for illustrating physical components of the apparatus for recommending customized cosmetics in FIG. 1.

FIG. 3 is a block diagram for illustrating functional components of the apparatus for recommending customized cosmetics in FIG. 1.

FIG. 4 is a flowchart for showing a method of recommending customized cosmetics based on artificial intelligence according to an embodiment of the present disclosure.

FIG. 5 is an example view showing how skin types are classified by the apparatus for recommending customized cosmetics according to an embodiment of the present disclosure.

FIG. 6 is an example view showing a process in which the apparatus for recommending customized cosmetics recommends customized cosmetics based on artificial intelligence according to an embodiment of the present disclosure.

### DETAILED DESCRIPTION

The description of the present disclosure is merely an example for structural or functional explanation, and the scope of the present disclosure should not be construed as being limited to the embodiments set forth herein. That is, the embodiments are to be construed as being variously embodied and having various forms, so that the scope of the present invention should be understood to include equivalents capable of realizing technical ideas. Also, the purpose or effect of the present invention should not be construed as limiting the scope of the present invention, since it does not mean that a specific embodiment should include all or only such effect.

Meanwhile, the meaning of the terms described in the present application should be understood as follows.

The terms “first”, “second”, and the like are intended to distinguish one element from another, and the scope of the right should not be limited by these terms. For example, the first component may be referred to as a second component, and similarly, the second component may also be referred to as a first component.

When one element is described as being “connected” to another element, it shall be construed as being connected or accessed to another element directly but also as possibly having yet another element in between. On the other hand, if one element is described as being “directly connected” to another element, it shall be construed that there is no other element in between. This is also true of other expressions for explaining a relationship between elements, i.e., “between” and “directly between” or “adjacent to” and “directly adjacent to”.

Unless clearly used otherwise, expressions in the singular number include a plural meaning. In the present description, an expression such as “comprising”, “including”, or “having” is intended to designate a characteristic, a number, a step, an operation, an element, a part or combinations thereof, and shall not be construed to preclude any presence or possibility of one or more other characteristics, numbers, steps, operations, elements, parts or combinations thereof.

Identification codes (e.g., a, b, and c) of each step are merely used for better comprehension and ease of description, not indicating a specific order of the steps, and the steps may be performed in a different order from a described order, unless clearly limited otherwise. Specifically, the steps may be performed in the same order as the described order, may substantially simultaneously be performed, or may be performed in the reverse order.

Unless otherwise defined, all terms used herein have the same meaning as how they are generally understood by those of ordinary skill in the art to which the disclosure pertains. Any term that is defined in a general dictionary shall be construed to have the same meaning in the context of the relevant art, and, unless otherwise defined explicitly, shall not be interpreted to have an idealistic or excessively formalistic meaning.

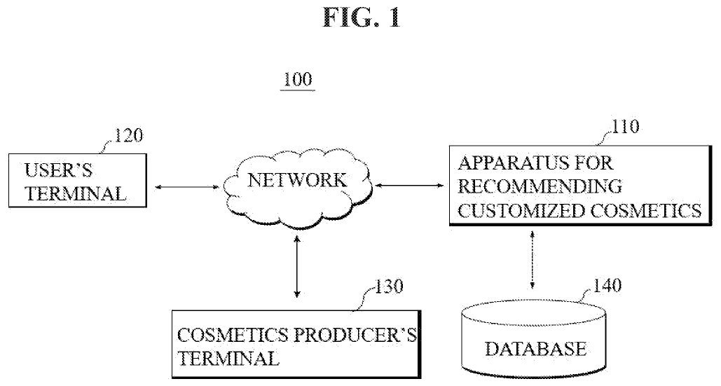

FIG. 1 shows a system for recommending customized cosmetics based on artificial intelligence according to an embodiment of the present disclosure.

Referring to FIG. 1, a system for recommending customized cosmetics based on artificial intelligence 100 may include an apparatus for recommending customized cosmetics based on artificial intelligence 110 (hereinafter, referred to as an “apparatus for recommending customized cosmetics”), a user's terminal 120, a cosmetics producer's terminal 130, and a database 140.

The apparatus for recommending customized cosmetics 110 may be formed as a server corresponding to computers or programs capable of recommending customized cosmetics optimized for skin condition of each individual user based on artificial intelligence (AI). According to an embodiment of the present disclosure, the apparatus for recommending customized cosmetics 110 may analyze information about a user's skin to determine base cosmetics and customized components based on the user's skin type and condition and recommend a formula based on the base and the customized components to the user, allowing the user to purchase or prepare customized cosmetics. Here, information on the user's skin may include an image of a target area on the user's skin and questionnaire data filled out by the user in relation to the target area on the skin. The apparatus for recommending customized cosmetics 110 may provide information about a formula of cosmetics tailored to the user's skin to a cosmetics producer, allowing the producer to produce customized cosmetics to be provided to the user.

The user's terminal 120 may correspond to a computing device for requesting a recommendation on customized cosmetics by the apparatus for recommending customized cosmetics 110, and may formed as a smartphone, a laptop, or a computer and as various devices such as a tablet PC, not being necessarily limited thereto. Here, the user's terminal 120 may be connected to the apparatus for recommending customized cosmetics 110 through a network. The user's terminal 120 may include a camera module for obtaining an image of a target area on a user's skin.

The cosmetics producer's terminal 130 may correspond to a computing device for receiving formula information about base cosmetics, components to be added, and the additional component content for customized cosmetics from the apparatus for recommending customized cosmetics 110. It may be possible for a cosmetics producer to produce customized cosmetics based on the received formula information and provide the customized cosmetics to a user who has requested purchases of the cosmetics.

The database 140 may be a storage device for storing information for a recommendation on customized cosmetics based on artificial intelligence by the apparatus for recommending customized cosmetics 110. According to an embodiment of the present disclosure, the database 140 may be operated in a cloud environment and connected to the apparatus for recommending customized cosmetics 110 through a network. According to an embodiment of the present disclosure, the database 140 may store skin analysis information based on multiple users' skin images, cosmetics base information based on the skin types, cosmetics formula information based on the skin conditions, etc., and may store information collected or processed in various forms while the apparatus for recommending customized cosmetics 110 is recommending customized cosmetics, not being necessarily limited thereto.

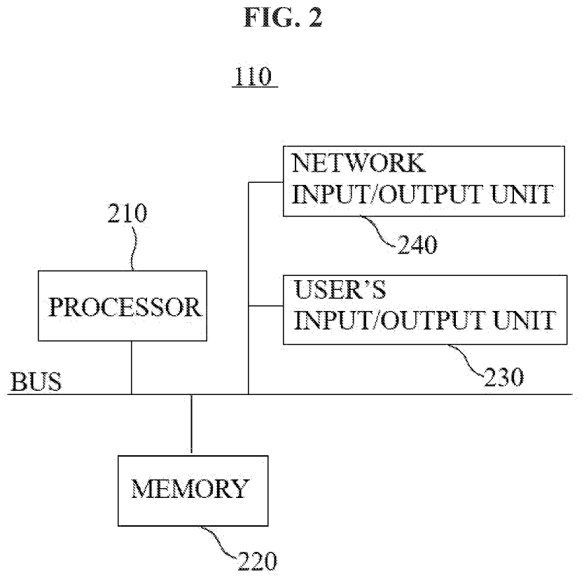

FIG. 2 is a block diagram for illustrating physical components of the apparatus for recommending customized cosmetics in FIG. 1.

Referring to FIG. 2, the apparatus for recommending customized cosmetics 110 may include a processor 210, a memory 220, a user's input/output unit 230, and a network input/output unit 240.

It may be possible for the processor 210 to recommend customized cosmetics based on artificial intelligence, manage the memory 220 that is read or written in the process, and schedule a synchronization time between a volatile memory and a non-volatile memory in the memory 220. It may be possible for the processor 210 to control the overall operation of the apparatus for recommending customized cosmetics 110 and control data flow between the memory 220, the user's input/output unit 230, and the network input/output unit 240 by being electrically connected to them. The processor 210 may be formed as a central processing unit (CPU) of the apparatus for recommending customized cosmetics 110.

The memory 220 may include an auxiliary memory formed as a non-volatile memory such as a solid state disk (SSD) or a hard disk drive (HDD) and used to store overall data required for the apparatus for recommending customized cosmetics 110 and a main memory formed as a volatile memory such as a random access memory (RAM).

The user's input/output unit 230 may include an environment for receiving information input by a user and an environment for outputting specific information to the user, and may include, for example, an input device including an adapter such as a mouse, a trackball, a touch pad, a graphics tablet, a scanner, a touch screen, a keyboard, or a pointing device and an output device including an adapter such as a monitor. According to an embodiment of the present disclosure, the user's input/output unit 230 may correspond to a computing device connected through a remote connection, and, in that case, the apparatus for recommending customized cosmetics 110 may be operated as a server.

The network input/output unit 240 may include at least an environment for connection with the apparatus for recommending customized cosmetics 110, and may include, for example, an adapter for a local area network) (LAN) communication. According to an embodiment of the present disclosure, the apparatus for recommending customized cosmetics 110 may be connected to the user's terminal 120, the cosmetics producer's terminal 130, or the database 140 through the network input/output unit 240.

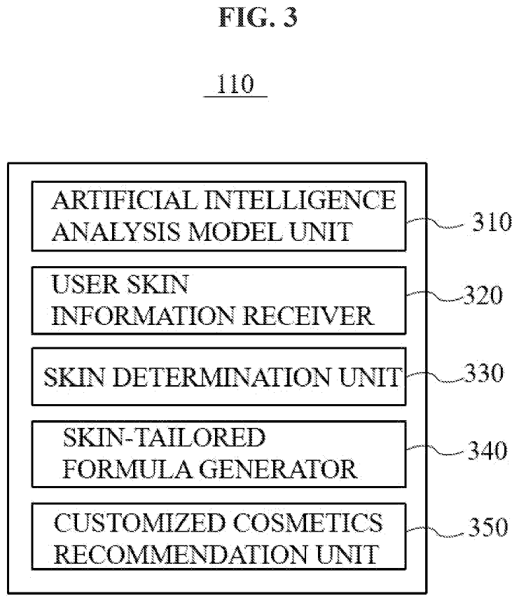

FIG. 3 is a block diagram for illustrating functional components of the apparatus for recommending customized cosmetics in FIG. 1.

Referring to FIG. 3, the apparatus for recommending customized cosmetics 110 may include an artificial intelligence analysis model unit 310, a user skin information receiver 320, a skin determination unit 330, a skin-tailored formula generator 340, a customized cosmetics recommendation unit 350, and a controller (not shown).

It may be possible for the artificial intelligence analysis model unit 310 to build an artificial intelligence analysis model for skin diagnosis through artificial intelligence (AI) learning based on images of the skin. According to an embodiment of the present disclosure, it may be possible for the artificial intelligence analysis model unit 310 to collect big data related to images of each target area on the skin of users of various ages and build an artificial intelligence analysis model for skin diagnosis through deep learning based on the collected images. Here, it may be possible for the artificial intelligence analysis model unit 310 to learn how closely skin condition and skin type are related to each other using information on an input skin image, thereby learning how closely the skin type is related to at least one of the skin condition and the information on the skin image.

It may be possible for the artificial intelligence analysis model unit 310 to build an artificial intelligence analysis model for skin diagnosis by learning the correlation between a skin image, skin type, and skin condition through a neural network. For example, the artificial intelligence analysis model unit 310 may label training images as “images with oil value 1, dead skin value 3, dandruff value 2, and sensitivity value 0” and learn each of hundreds of thousands of images to build an artificial intelligence analysis model for skin diagnosis. In this case, the values of oil, dead skin, dandruff, and sensitivity may be determined as one of 0 to 3.

According to an embodiment of the present disclosure, unlike the one shown in FIG. 3, the artificial intelligence analysis model unit 310 may be formed as an external system of the apparatus for recommending customized cosmetics 110 as an independent module, and may interoperate with the apparatus for recommending customized cosmetics 110.

The user skin information receiver 320 may receive information on a user's skin for recommendation on customized cosmetics from the user's terminal 120. To this end, the user skin information receiver 320 may provide a dedicated interface for inputting the information on the user's skin through the user's terminal 120. For example, the user's terminal 120 may install and execute a dedicated application, and the dedicated interface for inputting the information on the user's skin may be provided through the dedicated application. Thereafter, when the information on the user's skin is input, the user's terminal 110 may transmit the information to the user skin information receiver 320.

According to an embodiment of the present disclosure, the user skin information receiver 320 may receive a user's skin image and questionnaire data as information about the user's skin. Here, the user's skin image may be obtained by photographing a target area on the skin, such as the user's scalp, face, or body, using a camera provided in the user's terminal 120 or an independent camera. The information on the skin image may be used to objectively analyze the user's skin type and skin condition, such as dryness and oiliness of the skin, pores, skin tone, shine, troubles, blemishes, moles, freckles, and wrinkles. The questionnaire data may be obtained by providing a questionnaire containing multiple survey items asking about the user's gender, age, skin type, characteristics, concerns, lifestyle habits, etc. to the user's terminal 120 and receiving responses thereto. Because an individual's subjective living environment, such as enjoying greasy and sweet foods, lack of sleep, and frequent outdoor activities involving exposure to ultraviolet rays, may be a factor in determining skin condition, the user skin information receiver 320 may receive questionnaire data on a user's skin obtained by a questionnaire. The questionnaire data on a user's skin may be used to subjectively analyze the user's skin type and skin condition. The questionnaire data may include subjective opinions such as personal tastes or needs regarding cosmetics, such as a user's preferred scent or feeling of using the product.

The skin determination unit 330 may determine a user's skin type and skin condition by analyzing information about the user's skin. According to an embodiment of the present disclosure, the skin determination unit 330 may determine a candidate group of a user's skin type based on questionnaire data among information on the user's skin and determine the user's exact skin type and skin condition based on a skin image among the information on the user's skin. Specifically, the skin determination unit 330 may determine a candidate group of the user's skin type by analyzing the questionnaire data on the user's skin. According to an embodiment of the present disclosure, the skin determination unit 330 may perform natural language processing on questionnaire data on a user's skin and analyze the user's subjective skin condition from the questionnaire data to determine at least one candidate group of skin types. The skin determination unit 330 may set a target area on the skin based on questionnaire data and determine a candidate group of skin types based on the subjective condition of the set target area on the skin. Here, a target area on the skin is subdivided into scalp, face, and body, but is not limited thereto and may be subdivided into one or more. Skin type may be subdivided according to a target area on the skin. For example, types of scalps may be subdivided into dry, oily, sensitive, and hair loss, and types of face or body may be subdivided into dry, oily, combination, sensitive, and normal skin, not being necessarily limited thereto. The skin determination unit 330 may analyze external and internal factors, lifestyle habits, nutritional status of the skin, etc. that affect skin condition based on questionnaire data. In addition, the skin determination unit 330 may analyze a user's subjective opinion regarding cosmetics based on questionnaire data on the user's skin on which natural language processing has been performed. A user's subjective opinion may include personal preferences, experience, reasons for use, purchase conditions, design, preferred scent, etc. regarding cosmetics.

The skin determination unit 330 may analyze a user's skin image to determine a final skin type and skin condition of the skin type from a candidate group of skin types. In this case, the skin determination unit 330 may perform preprocessing to recognize a target area on the skin before analyzing the image. The skin determination unit 330 may perform binarization, color smoothing, histogram equalization, noise removal, etc. as preprocessing for a skin image. The skin determination unit 330 may perform preprocessing on a received skin image and carry out analysis with the preprocessed image.

The skin determination unit 330 may analyze information on a received image of a user's skin based on a pre-constructed artificial intelligence analysis model for skin diagnosis through the artificial intelligence analysis model unit 310 to determine skin type and skin condition from a candidate group of skin types. For example, the skin determination unit 330 may apply a received image of a user's skin to an artificial intelligence analysis model for skin diagnosis and determine skin type and skin condition based on result values including values of oil, dead skin cells, dandruff, and sensitivity. According to an embodiment of the present disclosure, the skin determination unit 330 may diagnose skin condition for multiple items for each target area on the skin and determine skin type. For example, as shown in Table 1 below, the items for analyzing and diagnosing scalp skin may be subdivided into five categories: oil, dandruff, dead skin cells, sensitivity, and hair loss; the items for analyzing and diagnosing facial skin may be subdivided into four categories: oil, whitening, skin sensitivity, and wrinkles; and the items for analyzing and diagnosing body skin may be subdivided into three categories: oil, whitening, and skin sensitivity. Whitening may include pigmentation.

TABLE 1

| Skin Area | Skin Diagnosis |
|---|---|
| Scalp | oil, dandruff, dead skin cells, sensitivity, and hair loss |
| Face | oil, whitening, skin sensitivity, and wrinkles |
| Body | oil, whitening, and skin sensitivity |

In the case of scalp, the skin determination unit 330 may classify oil, dandruff, dead skin cells, sensitivity, and hair loss into four grades and determine skin condition of scalp in 1,024 cases (=4×4×4×4×4).

According to an embodiment of the present disclosure, in the case of oil, the skin determination unit 330 may calculate oil scores based on questionnaire data or measure oil values using an algorithm for measuring an oil value. Here, when measuring an oil value with the algorithm for measuring an oil value, the skin determination unit 330 may extract skin features including color and texture of a target area on the skin from a pre-processed skin image, and may determine a value of skin oil by inputting the skin features into a regression analysis function prepared in advance based on the relationship between the extracted skin features and the oil content of the skin. The regression analysis function may be prepared in advance by learning the relationship between skin features in a skin image and the oil content of the skin measured by an oil meter.

The skin-tailored formula generator 340 may determine skin-customized ingredients based on skin type and skin condition to create a skin-customized formula. According to an embodiment of the present disclosure, the skin-tailored formula generator 340 may determine ingredients to be added to base cosmetics and the additional ingredient content based on skin condition. In this case, the skin-tailored formula generator 340 may reflect a user's subjective opinion and determine the emotional expression that customized cosmetics can give to the user. Here, the emotional expression of cosmetics may correspond to emotional items of cosmetics that can satisfy a user's emotions, and may vary depending on individual users' taste.

According to an embodiment of the present disclosure, the skin-tailored formula generator 340 may vary base cosmetics and additional ingredients, considering skin type and skin condition based on image-based AI skin diagnosis. Here, the image-based AI skin diagnosis may be divided into various categories as shown in Table 1 above. The skin-tailored formula generator 340 may subdivide additional cosmetic ingredients based on skin diagnosis. Based on skin condition, the skin-tailored formula generator 340 may select at least one additional ingredient from additional cosmetic ingredients subdivided based on skin diagnosis and determine the additional ingredient content.

The customized cosmetics recommendation unit 350 may recommend at least one of existing cosmetic products as base cosmetics based on skin type, and may transmit information about user-customized cosmetics to the user's terminal 120 by combining a skin-customized formula with the base. The existing cosmetic products may be pre-registered by setting skin types to which they can be applied. For example, Cosmetic A may be set as base cosmetics that can be recommended for skin types in the range of 2 to 3 for oiliness, 1 to 2 for whitening, and 1 to 3 for wrinkles. According to an embodiment of the present disclosure, the customized cosmetics recommendation unit 350 may recommend at least one existing cosmetic product set in a range suitable for a user's skin type as a base cosmetic product among pre-registered existing cosmetic products, and may recommend an existing cosmetic product set in a close range as a base cosmetic product when there are no cosmetic products set in a range suitable for the user's skin type.

The customized cosmetics recommendation unit 350 may transmit information on a skin-tailored formula to the cosmetics producer's terminal 130 upon a user's request in the process of transmitting information for recommending an existing cosmetic product suitable for the user's skin type as base cosmetics and the information on the skin-tailored formula to be added to the recommended base cosmetics to the user's terminal 110, allowing a cosmetics producer to provide a customized service for cosmetics to the user. In this case, the customized cosmetics recommendation unit 350 may transmit information on a formula including base cosmetics, additional ingredients, and the additional ingredient content, along with information on the emotional expression of the cosmetics, including scent and feeling of use, to the cosmetics producer's terminal 130, so that it may be possible for cosmetics producers to produce customized cosmetics that have ingredients and efficacy suited to users' skin and satisfy the users' sensibilities and provide the produced cosmetics to the users. In other words, it may be possible for cosmetics producers to provide users with personalized cosmetics prepared by mixing active ingredients with base cosmetics according to skin-tailored formulas. Otherwise, the cosmetics producers may provide users with ampoule with active ingredients added according to skin-tailored formulas along with base cosmetics, allowing the users to mix and use them themselves. Here, ampoule in quantity based on a formulation ratio may be provided to a user so that the user can mix and use them without difficulty. According to an embodiment of the present disclosure, the customized cosmetics recommendation unit 350 may provide a customized service for cosmetics to a user in the manner that personalized cosmetics prepared by mixing active ingredients with base cosmetics are provided or in the manner that base cosmetics and a separate ampoule containing active ingredients are provided for the user to mix them, and the manner in which the service is provided may be determined depending on the user's choice.

It may be possible for the controller (not shown) to control the overall operation of the apparatus for recommending customized cosmetics 110 and control data flow between the artificial intelligence analysis model unit 310, the user skin information receiver 320, the skin determination unit 330, the skin-tailored formula generator 340, and the customized cosmetics recommendation unit 350.

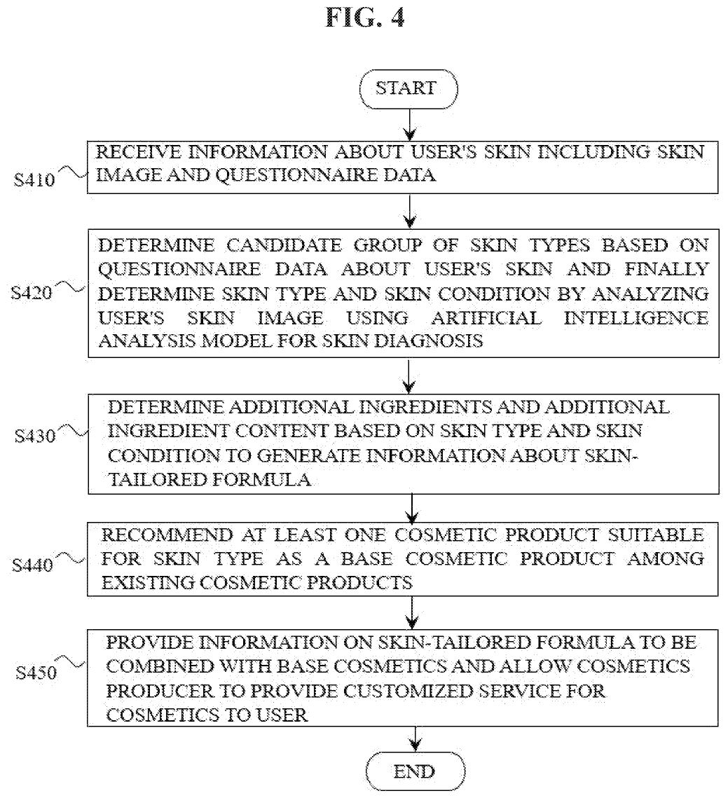

FIG. 4 is a flowchart for showing a method of recommending customized cosmetics based on artificial intelligence according to an embodiment of the present disclosure.

Referring to FIG. 4, in the artificial intelligence-based method of recommending customized cosmetics (hereinafter, referred to as a “method of recommending customized cosmetics”), the user skin information receiver 320 may receive information about a user's skin including the user's skin image and questionnaire data at S410.

In the method of recommending customized cosmetics, the skin determination unit 330 may determine a candidate group of skin types based on questionnaire data about a user's skin, and may finally determine the user's skin type and skin condition by analyzing the user's skin image using an artificial intelligence analysis model for skin diagnosis at S420. Here, the artificial intelligence analysis model for skin diagnosis may be pre-built by the artificial intelligence analysis model unit 310, and the skin determination unit 330 may analyze a user's skin image in conjunction with the artificial intelligence analysis model unit 310.

In the method of recommending customized cosmetics, the skin-tailored formula generator 340 may determine additional ingredients and the additional ingredient content based on skin type and skin condition to generate information about a skin-tailored formula at S430.

In the method of recommending customized cosmetics, the customized cosmetics recommendation unit 350 may recommend at least one existing cosmetic product suitable for a user's skin type as base cosmetics among existing cosmetic products at S440. In the method of recommending customized cosmetics, the customized cosmetics recommendation unit 350 may provide information on a skin-tailored formula to be combined with base cosmetics, and may allow a cosmetics producer to provide a customized service for cosmetics to a user upon the user's request at S450.

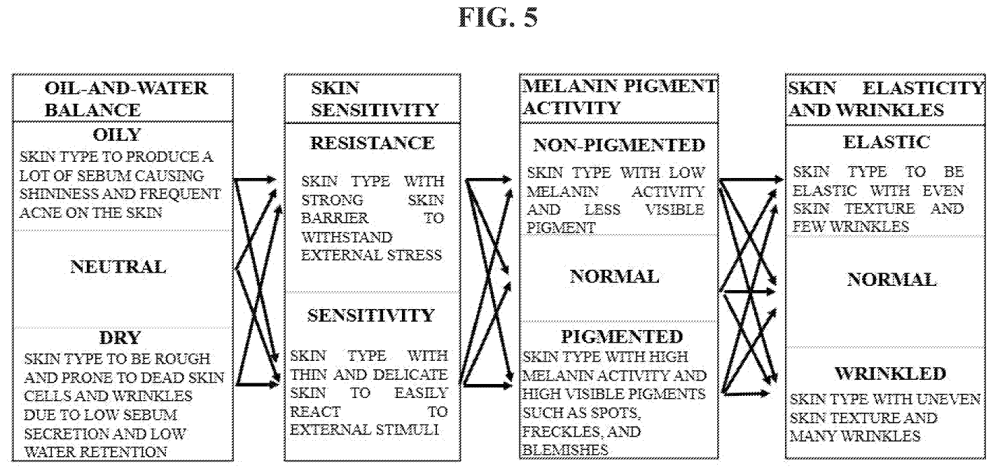

FIG. 5 is an example view showing how skin types are classified by the apparatus for recommending customized cosmetics according to an embodiment of the present disclosure.

Referring to FIG. 5, when a target skin is a face, skin type may be classified according to the state of oiliness, whitening, skin sensitivity, and wrinkles. According to an embodiment of the present disclosure, the apparatus for recommending customized cosmetics 110 may classify the state of oiliness into three levels: oily, neutral, and dry according to oil-and-water balance, may classify the state of skin sensitivity into two levels: resistance and sensitivity, may classify the state of whitening into three levels: non-pigmented, normal, and pigmented according to melanin pigment activity, and may classify the state of wrinkles into three levels: elastic, normal, and wrinkled according to skin elasticity and wrinkles. The apparatus for recommending customized cosmetics 110 may classify oiliness, whitening, skin sensitivity, and wrinkles of facial skin into levels, and may classify skin condition into 54 cases (=3×2×3×3) by combining their levels. Accordingly, the apparatus for recommending customized cosmetics 110 may recommend and provide user-centered customized cosmetics by diagnosing a user's skin condition in detail.

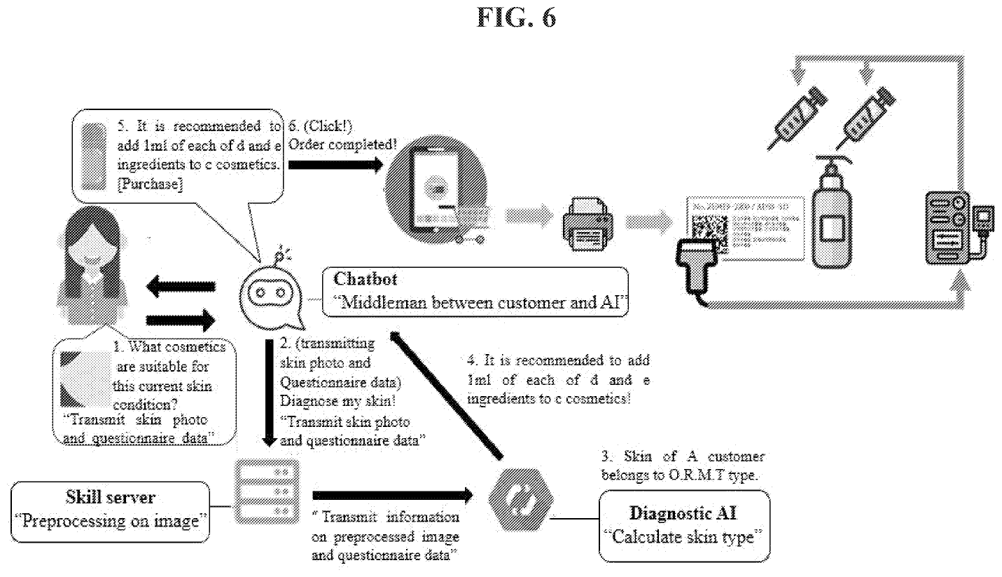

FIG. 6 is an example view showing a process in which the apparatus for recommending customized cosmetics recommends customized cosmetics based on artificial intelligence according to an embodiment of the present disclosure.

Referring to FIG. 6, it may be possible for a user to transmit skin photos and questionnaire data to the apparatus for recommending customized cosmetics 110 through the user's terminal 120 and request recommendations for cosmetics based on the user's current skin condition. Between the apparatus for recommending customized cosmetics 110 and the user's terminal 120, a process in which a user can easily diagnose his or her skin and receive recommendations for skin-tailored cosmetics may be carried out by a chatbot. The apparatus for recommending customized cosmetics 110 may perform skin diagnosis when skin photos and questionnaire data are transmitted from the user's terminal 120. In this case, the apparatus for recommending customized cosmetics 110 may perform preprocessing on skin images and diagnose information on the preprocessed images and questionnaire data through an AI analysis engine to calculate a user's skin type. The apparatus for recommending customized cosmetics 110 may recommend, to a user, ingredients to be added to base cosmetics and the additional ingredient content based on the user's skin type. Here, when a user makes a request for a purchase of cosmetics while the apparatus for recommending customized cosmetics 110 is recommending cosmetics, the apparatus for recommending customized cosmetics 110 may place an order for an existing cosmetic product suitable for a recommended formula or may provide a customized service for cosmetics by transmitting formula information to a cosmetics producer.

The present disclosure has been described above with reference to its desired embodiments, but a person having ordinary skill in the art would understand that the present disclosure can be modified and varied within the technology and the scope of the present disclosure as set forth in the claims below.

What is claimed is:

1. An apparatus for recommending customized cosmetics based on artificial intelligence, comprising:
a user skin information receiver for receiving information about a user's skin including a skin image and questionnaire data;
a skin determination unit for determining skin type and skin condition by analyzing information about the user's skin;
a skin-tailored formula generator for determining customized ingredients to be added and the additional ingredient content based on the skin type and the skin condition to generate information about a skin-tailored formula; and
a customized cosmetics recommendation unit for providing information about customized cosmetics that recommends at least one of existing cosmetic products pre-registered for each skin type as a base cosmetic product based on the skin type and provides information on user-customized cosmetics by combining a skin-tailored formula with the recommended base cosmetic product.

2. The apparatus of claim 1, wherein the skin determination unit determines at least one candidate group of skin types by analyzing a user's subjective skin condition based on the questionnaire data and finally determines the user's skin type and skin condition by analyzing the user's objective skin condition based on the skin image.

3. The apparatus of claim 2, wherein, based on an artificial intelligence analysis model for skin diagnosis that has been pre-built by artificial intelligence learning based on a skin image, the skin determination unit analyzes the received skin image for oiliness, dandruff, dead skin cells, erythema, and hair loss when a target skin is scalp, for oiliness, whitening, skin sensitivity, and wrinkles when a target skin is a face, and for oiliness, whitening, and skin sensitivity when a target skin is a body.

4. The apparatus of claim 1, wherein the customized cosmetics recommendation unit sets the range of skin types to which existing cosmetic products can be applied, recommends at least one existing cosmetic product set in a range suitable for a user's skin type as a base cosmetic product among pre-registered existing cosmetic products, and recommends an existing cosmetic product set in a close range as a base cosmetic product when there are no cosmetic products set in a range suitable for the user's skin type.

5. The apparatus of claim 4, wherein the customized cosmetics recommendation unit provides a user with customized cosmetics prepared by mixing active ingredients with a recommended base cosmetic product based on the information on the skin-tailored formula or provides the base cosmetic product and ampoule containing the active ingredients for the user to mix them.

6. A method of recommending customized cosmetics based on artificial intelligence, comprising:
receiving information about a user's skin including a skin image and questionnaire data for the user;
determining a candidate group of skin types based on the questionnaire data and analyzing the received skin image based on a pre-constructed artificial intelligence analysis model for skin diagnosis to finally determine skin type and skin condition;
generating information on a skin-tailored formula by determining additional ingredients and the additional ingredient content based on the finally determined skin type and skin condition; and
recommending at least one of pre-registered existing cosmetic products for each skin type as a base cosmetic product based on the skin type and providing the information about the skin-tailored formula to be combined with the recommended base cosmetic product, allowing a cosmetics producer to provide a user with a customized service for cosmetics.
